(19) United States
(12) Patent Application Publication
(10) Pub. No.: US 2022/0355233 A1
(43) Pub. Date: Nov. 10, 2022

(54) PATHOGEN SURVEILLANCE SYSTEMS AND CORRESPONDING METHODS

(71) Applicant: Joseph J.K. Ma, North York (CA)

(72) Inventor: Joseph J.K. Ma, North York (CA)

(21) Appl. No.: 17/565,172

(22) Filed: Dec. 29, 2021

Related U.S. Application Data
(60) Provisional application No. 63/184,606, filed on May 5, 2021.

(51) Int. Cl. B01D 46/42; B01D 46/00; A61L 9/20; B01D 46/62; G01N 1/22

(52) U.S. Cl. CPC B01D 46/429; B01D 46/0028; A61L 9/20; B01D 46/62; G01N 1/2247; B01D 2279/65; A61L 2209/111; B01D 2279/50; A61L 2209/14

(57) ABSTRACT

A method of monitoring indoor environments for aerosolized pathogens includes: (a) receiving a pathogen status for each of a plurality of aerosol samples collected by a plurality of corresponding air treatment devices; (b) determining whether the pathogen status for each aerosol sample changes a safety status of a corresponding indoor environment from which the aerosol sample was collected; and (c) in response to determining a change in the safety status, generating a notification associated with the change in the safety status for the corresponding indoor environment.

### CROSS-REFERENCE TO RELATED APPLICATIONS

This application claims the benefit of U.S. Provisional Application No. 63/184,606 filed May 5, 2021, the entirety of which is hereby incorporated herein by reference.

### FIELD

The specification relates generally to pathogen surveillance systems and corresponding methods for monitoring indoor environments for aerosolized pathogens.

### BACKGROUND

Certain diseases can transmit via aerosolized pathogens (e.g., viruses, bacteria, and spores). This type of transmission is of particular concern for indoor and/or poorly ventilated environments where aerosols can linger for relatively long periods of time.

### SUMMARY

The following summary is intended to introduce the reader to various aspects of the applicant's teaching, but not to define any invention.

According to some aspects, a pathogen surveillance system configured to monitor indoor environments for aerosolized pathogens includes: (a) one or more air treatment devices for treating air in corresponding indoor environments. Each air treatment device includes: (i) at least one air flow passage extending through the device; (ii) an air circulation unit operable to urge flow of air through the air flow passage; (iii) an air filtration unit in the air flow passage for filtering the air flowing through the air flow passage; (iv) a sterilization unit operable to sterilize the air flowing through the air flow passage; and (v) an aerosol collection unit configured to collect an aerosol sample from the air flowing into the air flow passage. The system further includes (b) an air monitoring system includes a processor operable to: receive a pathogen status for the aerosol sample collected by the aerosol collection unit; determine whether the pathogen status of the aerosol sample changes a safety status of a corresponding indoor environment from which the aerosol sample was collected; and in response to determining a change in the safety status, generate a notification associated with the change in the safety status for the indoor environment.

In some examples, generating the notification includes updating a visual representation of the indoor environment based on the safety status change.

In some examples, the visual representation includes a portion of a map corresponding to a location of the indoor environment. In some examples, the map includes topographic features indicative of an elevation of the location.

In some examples, the one or more air treatment devices include a plurality of the air treatment devices positionable in different locations, and wherein the visual representation include portions of a map corresponding to the different locations.

In some examples, the visual representation includes a 3-dimensional representation of a location of the indoor environment within a building.

In some examples, each air treatment device includes a user tracking unit for collecting user tracking data associated with individuals within a detection range of the air treatment device.

In some examples, generating the notification includes: identifying one or more individuals affected by the change in the safety status for the indoor environment based on the user tracking data collected by the user tracking unit; and generating an alert for transmission to the one or more individuals regarding the change in the safety status.

In some examples, the system further includes a diagnostic system operable to detect pathogens in the aerosol sample for generation of the pathogen status.

In some examples, each air treatment device includes a corresponding diagnostic device of the diagnostic system, the diagnostic device operable to detect pathogens in the aerosol sample collected by the air treatment device.

In some examples, the diagnostic device includes at least one of a particle detector and an antigen detector.

In some examples, the diagnostic system is operable to detect pathogens based on nucleic acid sequences.

In some examples, the filtration unit includes one or more air filters extending across the air flow passage.

In some examples, the sterilization unit is operable to sterilize the filtration unit.

In some examples, the aerosol collection unit is upstream of the filtration unit.

In some examples, the aerosol collection unit includes an aerosol capture medium extending only partially across the air flow passage to permit air flow around the aerosol collection unit.

In some examples, the aerosol collection unit includes a plurality of aerosol sampling elements configured to collect corresponding aerosol samples at different times.

In some examples, the monitoring system receives the pathogen status for the aerosol sample from the one or more air treatment devices via a network.

In some examples, the monitoring system receives the pathogen status for the aerosol sample from a remote computing device via a network.

In some examples, the air circulation unit includes one or more fans.

In some examples, the sterilization unit includes an ultraviolet C (UVC) source for UVC irradiation.

In some examples, each air treatment device is mobile to facilitate movement to different locations.

In some examples, each air treatment device includes a processor and a drive system operable by the processor to enable autonomous movement of the air treatment device.

In some examples, the system further includes a mobile duct system including one or more ducts connectable between an outlet of the air flow passage and an external environment outside the indoor environment for negatively pressurizing the indoor environment.

In some examples, the one or more air treatment devices include a plurality of air treatment devices, and each air treatment device includes a wireless communication unit to enable wireless data transfer and automated coordination of the plurality of air treatment devices.

In some examples, each air treatment device includes a sensor system having one or more environmental sensors for sensing environmental conditions associated with pathogen exposure risk of the indoor environment, and wherein the processor of the monitoring system is operable to determine whether to change the safety status for the indoor environment based at least in part on the environmental conditions.

In some examples, the environmental conditions include at least one of temperature, humidity, and barometric pressure.

According to some aspects, a method of monitoring indoor environments for aerosolized pathogens includes: (a) receiving a pathogen status for each of a plurality of aerosol samples collected by a plurality of corresponding air treatment devices; (b) determining whether the pathogen status for each aerosol sample changes a safety status of a corresponding indoor environment from which the aerosol sample was collected; and (c) in response to determining a change in the safety status, generating a notification associated with the change in the safety status for the corresponding indoor environment.

In some examples, generating the notification includes updating a visual representation of the corresponding indoor environment based on the safety status change.

In some examples, the visual representation includes a portion of a map corresponding to a location of the corresponding indoor environment.

In some examples, each air treatment device includes a user tracking unit for collecting user tracking data associated with individuals within a detection range of the air treatment device. Generating the notification includes: identifying one or more individuals affected by the change in safety status based on user tracking data collected by a user tracking unit of the air treatment device; and generating an alert for transmission to the one or more individuals, the alert advising the one or more individuals of the change in the safety status.

In some examples, the method further includes determining the pathogen status through testing of the aerosol sample based on nucleic acid sequences.

In some examples, the method further includes, for each air treatment device, urging flow of air from the corresponding indoor environment through an aerosol collection unit positioned in an air flow passage of the air treatment device to collect the aerosol sample, and sterilizing the air flowing through the air flow passage downstream of the aerosol collection unit.

According to some aspects, a monitoring system for aerosolized pathogens includes a processor operable to: (a) receive a pathogen status for each of a plurality of aerosol samples collected by a plurality of corresponding air treatment devices; (b) determine whether the pathogen status for each aerosol sample changes a safety status of a corresponding indoor environment from which the aerosol sample was collected; and (c) in response to determining a change in the safety status, generate a notification associated with the change in the safety status for the corresponding indoor environment. In some examples, generating the notification comprises updating a visual representation of the corresponding indoor environment based on the safety status change. In some examples, the visual representation comprises a portion of a map corresponding to a location of the corresponding indoor environment.

### BRIEF DESCRIPTION OF THE DRAWINGS

The drawings included herewith are for illustrating various examples of apparatuses, systems, and methods of the present specification and are not intended to limit the scope of what is taught in any way. In the drawings:

FIG. 1 is a block diagram of an example pathogen surveillance system;

FIG. 2 is an illustration of an example indoor environment, showing air flow in the indoor environment while an example air treatment device of the system of FIG. 1 is in use;

FIG. 3 is an exploded perspective view of the air treatment device of FIG. 2;

FIG. 4 is a block diagram of the air treatment device of FIG. 2;

FIG. 5 is a perspective view of an example air treatment assembly for the system of FIG. 1;

FIG. 6 shows additional examples of air treatment devices for the system of FIG. 1;

FIG. 7 is a flowchart showing an example method for monitoring air in an indoor environment for aerosolized pathogens;

FIG. 8 is an example visual representation generated by the system of FIG. 1 for presenting safety status of a plurality of different indoor environments monitored by the system of FIG. 1; and

FIGS. 9A and 9B are diagrams showing the system of FIG. 1 in use for contact tracing in accordance with an example embodiment.

### DETAILED DESCRIPTION

Various apparatuses, systems, or processes are described herein to provide an example of an embodiment of each claimed invention. No embodiment described limits any claimed invention and any claimed invention may cover apparatuses, systems, or processes that differ from those described herein. The claimed inventions are not limited to apparatuses, systems, or processes having all of the features of any one apparatus, system, or process described below or to features common to multiple or all of the apparatuses, systems, or processes described. It is possible that an apparatus, system, or process described is not an embodiment of any claimed invention. Any invention disclosed in an apparatus, system, or process described that is not claimed in this document may be the subject matter of another protective instrument, for example, a continuing patent application, and the applicants, inventors, or owners do not intend to abandon, disclaim, or dedicate to the public any such invention by its disclosure in this document.

Improved ventilation within indoor environments can help reduce disease transmission caused by aerosolized pathogens. There are limitations to only improving ventilation. For example, traditional methods of improving ventilation often involve a time delay that is dependent on the effective air exchanges per hour of a given environment. The effective air exchanges of an environment are affected not only by HVAC systems, but also by the positioning of air entrance(s) and exit duct(s), and any obstructions within the environment (e.g., furniture, equipment, and/or architectural features). Also, only improving ventilation in environments that cannot easily ventilate to either the outside or to a larger space may be insufficient for eliminating aerosolized pathogens.

Regular infectious monitoring of individuals that congregate within indoor and/or poorly ventilated environments (e.g., sports arenas, schools, and/or work environments) can help reduce the transmission of disease. However, this type of infectious monitoring can in some cases require regular nasal swabs throughout the day, which can be uncomfortable and expensive. There is also a requirement of compliance among participants as well as those who are indirectly involved, such as visitors, audience members, maintenance workers, and/or other third-parties who may cross paths with participants.

Disclosed herein are various embodiments of air treatment devices operable to provide ultra-high flow ventilation in addition to disinfection and sterilization functionality, which can increase the rate of aerosol disinfection without requiring construction, external ventilation, or renovations of duct work. In some embodiments, the air treatment devices can be mobile to facilitate movement among different locations. In some embodiments, the air treatment devices can be used in operating rooms, clinical settings, gyms, a workplaces, rooms, buildings, cities, states, etc.

According to some aspects of the present disclosure, the air treatment devices can be integrated as part of a pathogen surveillance system for monitoring indoor environments for aerosolized pathogens. In some embodiments, the pathogen surveillance system includes one or more air treatment devices, a diagnostic system, and an air monitoring system. Each air treatment device is positionable in a corresponding indoor environment for treating air of the indoor environment and collecting aerosol samples from the air. The diagnostic system is operable to detect pathogens in the aerosol sample for generation of a pathogen status associated with the aerosol sample for the indoor environment. The air monitoring system is operable to receive the pathogen status for the aerosol sample; determine whether the pathogen status of the aerosol sample changes a safety status of a corresponding indoor environment from which the aerosol sample was collected; and in response to determining a change in the safety status, generate a notification associated with the change in the safety status for the indoor environment.

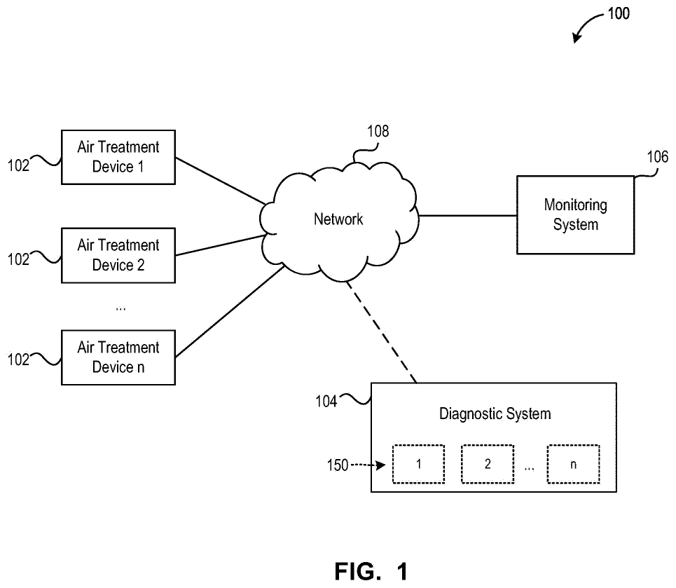

Referring to FIG. 1, a block diagram of an example pathogen surveillance system 100 is illustrated. In the example illustrated, the pathogen surveillance system 100 includes one or more air treatment devices 102, a diagnostic system 104, and a monitoring system 106. In the example illustrated, the air treatment devices 102, diagnostic system 104, and monitoring system 106 are shown in communication through a network 108.

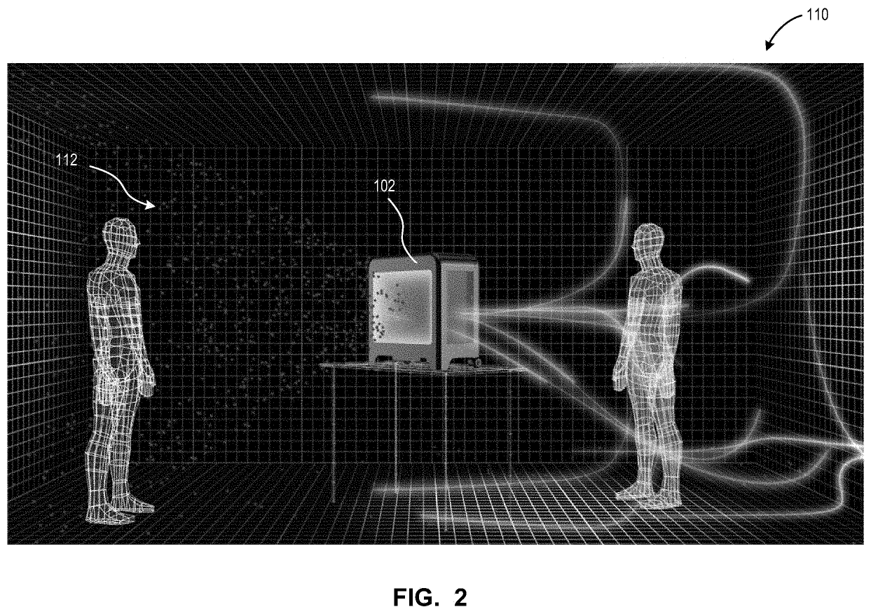

Referring to FIG. 2, each air treatment device 102 is positionable in a corresponding indoor environment 110 for treating air 112 in the indoor environment 110. Multiple air treatment devices 102 can be positioned in a common indoor environment (e.g. in the same room of a building), or the air treatment devices 102 can be positioned at different locations (e.g. different rooms, floors, buildings, cities, etc.).

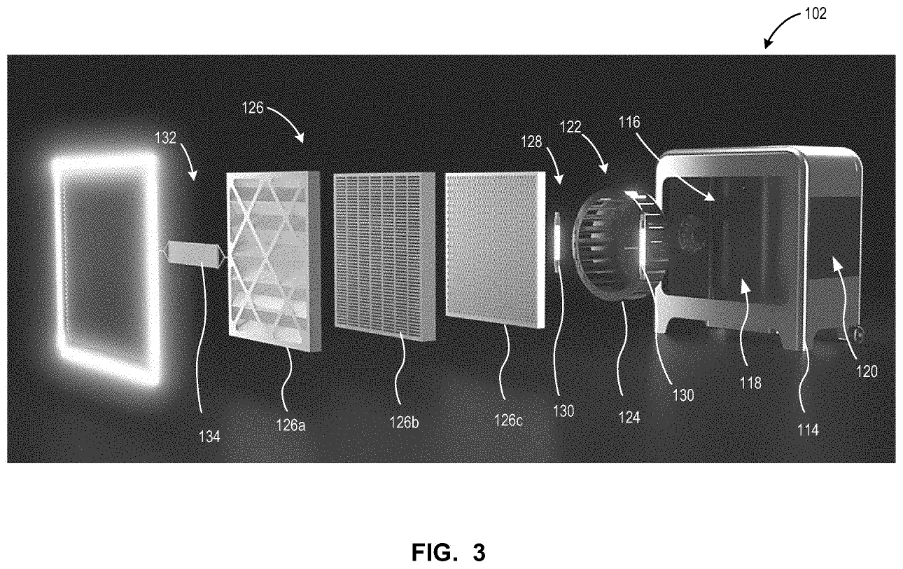

Referring to FIG. 3, in the example illustrated, each air treatment device 102 includes a casing 114 and at least one air flow passage 116 extending through the casing 114 between at least one inlet 118 for intake of air from the indoor environment and at least one outlet 120 for discharge of the air from the device 102 (e.g. either back into the indoor environment or vented to outside the indoor environment).

In the example illustrated, the air treatment device 102 has an air circulation unit 122 operable to urge flow of air through the air flow passage 116 (e.g. by suctioning air through the inlet 118 and discharging the air through the outlet 120). In the example illustrated, the air circulation unit 122 comprises one or more fans 124 (e.g. a centrifugal fan) in the casing 114. The air circulation unit can operate to generate relatively high flow rates, for example, in the range of 400 to 1000 CFM. When multiple air treatment devices 102 are used together (in series or in parallel) in a common indoor environment, an ultra-high effective flow rate in the range of, for example, 1200 to 3000 CFM may be achieved within the indoor environment. The air discharged from the outlet 120 can, in some embodiments, be designed to result in a laminar air flow. The air treatment device 102 can be set up so that the air flow back to the indoor environment is directed into a sterile field or used as a virtual shield to protect the sterile environment. In some embodiments, the air treatment device 102 can cool the air before it is discharged from the outlet 120. In some embodiments, the system 100 can include a mobile duct system having one or more ducts connectable between the outlet 120 of the air treatment device 102 and an external environment outside the indoor environment. This can allow for evacuation of air from the indoor environment for negatively pressurizing the indoor environment.

In the example illustrated, the air treatment device 102 includes an air filtration unit 126 in the air flow passage 116 (and casing 114) for filtering the air flowing through the air flow passage 116. In the example illustrated, the air filtration unit 126 is upstream of the air circulation unit 122. The air filtration unit 126 can include one or more air filters extending across the air flow passage 116. The air filters can comprise, for example, HEPA or Merv-17 equivalent air filters. In the example shown in FIG. 3, the filtration unit 126 includes a pre-filtration component 126a for capturing larger particles and a particulate filtration component 126b (e.g., a HEPA filter or Merv-17 equivalent air filter) for capturing fine particles. In the example shown in FIG. 3, the filtration unit 126 further includes an optional activated carbon component 126c for reducing odor from the air (and within the indoor environment). For some environments, the air treatment device 102 may not include/require an air filtration unit. For example, if the air is discharged from the air treatment device 102 to an external environment where the pathogens would be significantly diluted at high volumes, the filtration unit 126 (or one or more filtration sub-components) may be omitted. However, in environments in which the air is discharged back into the indoor environment or into another environment where individuals may be exposed to potential pathogens, or if the calculated dilution of the pathogens would be insufficient, the air treatment device 102 can include the filtration unit 126 to reduce pathogen exposure risk.

In the example illustrated, the air treatment device 102 includes a sterilization unit 128 operable to sterilize the air flowing through the air flow passage 116. In the example illustrated, the sterilization unit 128 is in the casing 114 downstream of the filtration unit 126 and upstream of the air circulation unit 122. When the filtration unit 126 is included, the sterilization unit 128 can operate to sterilize the filtration unit 126 and/or air passing through from the filtration unit 126. In the example illustrated, the sterilization unit comprises an ultraviolet C (UVC) source 130 for UVC irradiation. Irradiation in the UVC range can facilitate sterilization/disinfection by affecting nucleic acids (RNA and/or DNA) of pathogens. This mechanism can include the formation of thymine dimers in the RNA or DNA which prevents the replication of the DNA or RNA of the pathogen. Sources of such irradiation can include, for example, tube lighting, LED lighting, or Laser in UVC wavelength. Lasers can be native to the UVC range or can be frequency doubled or frequency quadrupled to the UVC wavelength. The air in the indoor environment can be irradiated as it flows through the air flow passage 116 past columns, and/or when external the air treatment device (as described below). In some examples, pathogens can be irradiated by UVC when captured on a filter of the filtration unit 126. In the example illustrated, the UVC source 130 is positioned within the casing 114 of the air treatment device 102 for irradiation of air flowing through the air flow passage 116, and is generally shielded from directly irradiating the indoor environment. In some modes of operation (described below), the shielding can be removed for direct UVC irradiation of the indoor environment including the air and surfaces external the casing 114 of the air treatment device 102. In some examples, the sterilization unit 128 can include a heat source for disabling and denaturing pathogens passing the heat source. The heat source can include, for example, heat generated as a byproduct of electronic or electromechanical devices (e.g., heat generated from batteries, ballasts, lights, electronics, inverters, chargers, etc.), and/or dedicated heating elements. In some examples, this can be augmented by dedicated heating elements past which the air is conducted when flowing through the air flow passage 116.

The air treatment devices 102 can be operable in different modes for treatment of air and/or surfaces within the indoor environment. In a first (filtration) mode, the air treatment device 102 can be in continuous operation for circulating air within the indoor environment through the filtration unit 126. This mode can increase the effective air exchange rate in the indoor environment and decrease the time for aerosolized pathogen disinfection. This mode can also help dislodge otherwise non-circulating lingering aerosols, and help to preserve a sterile field above the air flow level, and reduce contamination from below air flow level such as from floors, shoes, dust, etc. This mode may be appropriate when the indoor environment is in use (e.g., occupied by individuals, when clients are present, etc.).

In a second (filtration and sterilization) mode, the air treatment device 102 can operate so that the filtration unit 126 and the sterilization unit 128 are used simultaneously (with UVC shielding in place). In this mode, the air treatment device 102 can be in operation for circulating air within the indoor environment through the filtration unit 126 in addition to sterilization of the air flowing through the air flow passage 116 via operation of the sterilization unit 128. In this mode, the effective air exchange rate and rate of aerosolized pathogen disinfection within the indoor environment can be increased relative to the first mode. This mode may also be appropriate when the indoor environment is in use (e.g., during medical procedures within a sterile environment, when clients are present, etc.).

In a third (unshielded) mode, the air treatment device 102 can operate so that the filtration unit 126 and the sterilization unit 128 are used simultaneously, but with UVC shielding removed for direct UVC irradiation of the indoor environment external the casing 114 by the sterilization unit 128. This mode can utilize multiple methods of disinfection/sterilization (such as heat and UVC irradiation), and can directly sterilize external surfaces within the indoor environment with UVC irradiation to further increase the rate of pathogen disinfection. This mode can also directly disinfect non-circulating lingering aerosols exposed to the UVC irradiation, and help to preserve a sterile field of air flow. The third mode can be a timed mode, and can be used when individuals are not within the indoor environment/proximity of the air treatment device 102 due to lack of UVC shielding (e.g. after a medical procedure is completed and all individuals leave the room).

In the example illustrated, each air treatment device 102 further includes an aerosol collection unit 132 configured to collect an aerosol sample from air flowing into the air flow passage 116. In the examples illustrated, the aerosol collection unit 132 is positioned in the casing 114 at the inlet 118 and upstream of the filtration unit 126. In the example illustrated, the aerosol collection unit 132 includes an aerosol collection medium 134 in the air flow passage 116. In the example illustrated, the aerosol collection medium 134 extends only partially across the inlet 118 of the air flow passage 116 to permit air to flow around the aerosol collection unit 132 (so as not to significantly impede air flow through the air flow passage 116). The aerosol collection medium 134 can include filtration elements (e.g. filter paper) intended for capturing aerosol (including aerosolized pathogens). The aerosol collection medium can absorb moisture, and/or comprise electrostatically charged material for attracting water ions to facilitate aerosol collection. In some embodiments, the air treatment device 102 can operate such that the flow rate of the air flowing through the air flow passage 116 can result in the collection of aerosol samples from the equivalent of the local volume of air in the indoor environment every few minutes or less. In the example illustrated in FIG. 3, the aerosol collection unit 132 is shown with a single aerosol collection medium 134.

In some examples, the aerosol collection unit 132 can include a plurality of aerosol sampling elements (e.g. separate collections mediums, or different portions of a common collection medium) configured to collect corresponding aerosol samples at different times. For example, an array of the sampling elements can be positioned across the air flow passage, or a series of the sampling elements spaced apart from each other along the air flow passage 116. In some examples, each sampling element can have a barrier isolating a corresponding aerosol collection medium (or portion thereof) of the sampling element from air flowing into the air flow passage 116, and the barriers can be selectively releasable (e.g. removed, dissolved, translated, etc.) to expose corresponding aerosol collection media (or portions thereof) at different times for collecting aerosol samples at the different times. In some examples, the barriers can be releasable at predetermined time periods, and/or in response to specific environmental triggers detected by the air treatment device 102 (e.g. detection of a predetermined number of individuals having been in proximity to the device 102). After a corresponding sampling element has been exposed to the air flow for a time period to collect an aerosol sample, the sampling element can be isolated from the air flowing through the air flow passage 116 (e.g. by the same or another barrier, or by moving or removing the sampling element from the air flow passage 116) and associated with the time period for determining when the aerosol sample was collected.

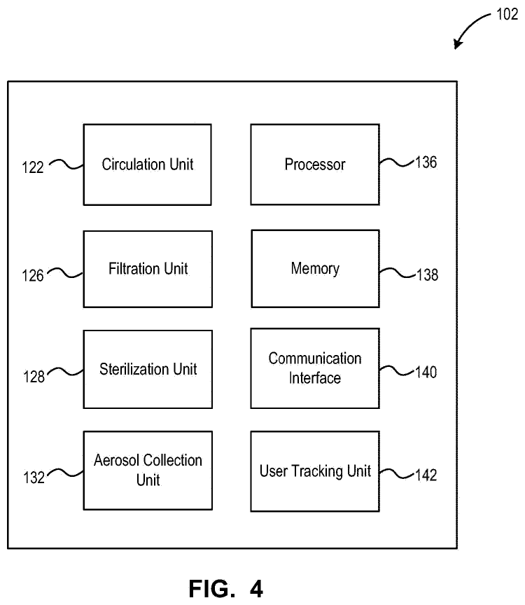

Referring to FIG. 4, in the example illustrated, the air treatment device 102 includes a processor 136, memory 138, a communication interface 140, and a user tracking unit 142. In some embodiments, the processor 136, memory 138, and the communication interface 140 can be combined together or further separated into one or more different components. The processor 136, memory 138, and communication interface 140 can be implemented in software or hardware, or a combination of software and hardware. The processor 136 may be any suitable processors, controllers or digital signal processors that can provide sufficient processing power depending on the configuration, purposes, and requirements of the air treatment device 102.

In the example illustrated, the user tracking unit 142 is operable to collect user tracking data associated with individuals within a detection range of the air treatment device 102. In some embodiments, the user tracking unit 142 can track the individuals by communicating to them via an application installed on each individual's mobile device, such as including but not limited to smart watches, smart bands, smart tags via various wireless transmissions including, but not limited to, Bluetooth, Wi-fi, microwave, ultra high frequency (UHF), and Near-Field Communication (NFC). The memory 138 can include RAM, ROM, one or more hard drives, one or more flash drives or some other suitable data storage elements such as disk drives, etc. The memory 138 can include one or more databases (not shown) for storing information relating to the operation of the air treatment device 102, such as data related to the aerosol samples and/or user tracking data.

The communication interface 140 can include any interface that enables the air treatment device 102 to communicate with other devices and systems. In some embodiments, the communication interface 140 can include at least one of a wireless communication interface, serial port, a parallel port, a USB port. The communication interface 140 may also include at least one of an Internet, Local Area Network (LAN), Ethernet, Firewire, modem or digital subscriber line connection. Various combinations of these elements may be incorporated within the communication interface 140. For example, the communication interface 140 may receive input from various input devices, such as a mouse, a keyboard, a touch screen, a thumbwheel, a track-pad, a track-ball, a card-reader, voice recognition software and the like depending on the requirements and implementation of the air treatment device 102. In some embodiments, the communication interface 140 can provide positioning data via various wireless communication implementations, such as, but not limited to, Global Positioning System (GPS), Bluetooth, NFC, UHF, and/or Light Detection and Ranging (LiDAR) technology.

In some embodiments, the air treatment devices 102 can communicate with each other (e.g. directly with each other through communication interfaces 140, and/or through the network 108). In some embodiments, the air treatment devices 102 can detect one or more other air treatment devices 102 within an environment via their communication interface 140 to determine a relative positioning of each other. For example, the air treatment devices 102 can communicate with each other to determine a relative position, and then transmit, via the communication interface 140, their positions to the monitoring system 106. In some embodiments, the air treatment devices 102 can determine their relative positioning using GPS or LiDAR, for example. In some embodiments, the air treatment devices 102 can also continuously monitor the position of itself and/or other air treatment devices 102 and send updates to the monitoring system 106 of corresponding locations periodically.

In some embodiments, the air treatment device 102 can include a drive system operable by the processor 136 to enable autonomous movement of the air treatment device 102. The drive system can include, for example, electrically driven wheels and a drive sensor system for scanning the environment to facilitate autonomous movement. For example, the air treatment device 102 can be operated by the processor 136 to follow an individual or a group of individuals to reduce their exposure to pathogens and to focus monitoring of their potential exposure to the pathogens. In another example, the air treatment device 102 can be operated to autonomously move between different areas within an indoor environment to sterilize air and/or collect aerosol samples from the different areas.

In some embodiments, more than one air treatment device 102 can be used simultaneously in the same environment. The air treatment devices 102 may be stationary, or one or more air treatment devices 102 may be mobile and include wheels (e.g. mounted to the casing 114) to facilitate movement of the devices. The air treatment devices can be autonomously operated by the processor 136 (e.g. through operation of corresponding drive systems of the air treatment devices). The air treatment devices 102 can communicate with each other for coordinating movement and operation of the system 100.

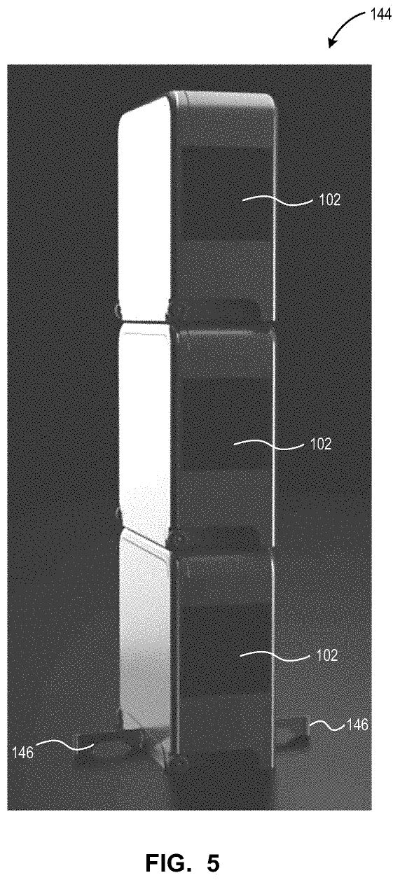

Referring to FIG. 5, in the example illustrated, an air treatment assembly 144 is illustrated. The air treatment assembly 144 includes a plurality of the air treatment devices 102 coupled together (stacked atop each other, in the example illustrated). In the example illustrated, a support structure 146 is coupled to a bottom one of the air treatment devices to provide support for the assembly 144. In the example illustrated, the support structure 146 comprises support legs extending from the bottom air treatment device 102. Other support structures can be used depending on requirements for the indoor environment. The air treatment assembly 144 can help increase system functionality (e.g., time required to eliminate pathogen risk can be reduced as the ventilation per hour is increased) without a need to increase the overall footprint of the air treatment devices 102. The air treatment assembly 144 can also enable incremental adjustment of the air flow to adapt to the indoor environment. When multiple air treatment devices 102 form the air treatment assembly 144, the air treatment devices 102 can be connected in series to obtain power from each other such that only one air treatment device 102 needs to have a direct power supply (either direct A/C current or a rechargeable battery). The air treatment assembly 144 can also provide for a plurality of the aerosol collection units 132 at the same indoor location (e.g. one or more in each air treatment device 102), and the aerosol collection units 132 can be configured to collect corresponding aerosol samples at different times (e.g. by initiating operation of corresponding air treatment devices 102 at different times).

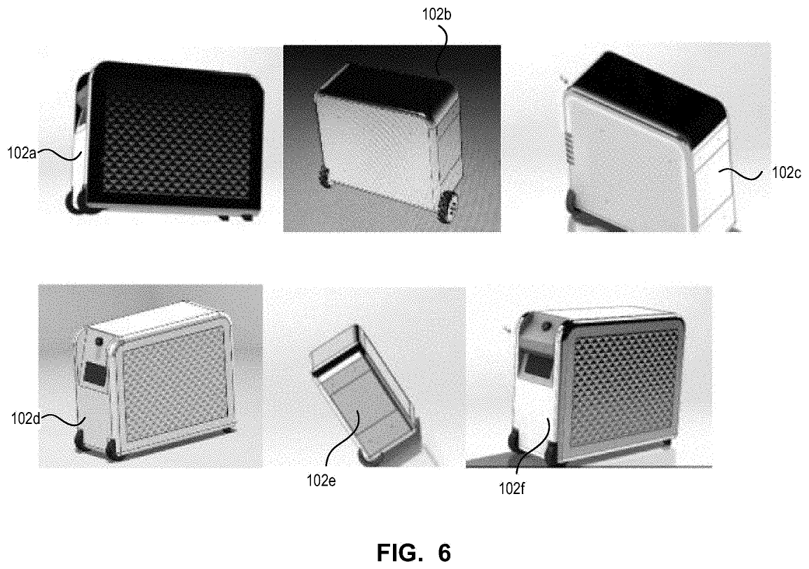

FIG. 6 shows other example air treatment devices 202a, 202b, 202c, 202d, 202e, and 202f.

Referring to FIG. 1, in the example illustrated, the diagnostic system 104 is operable to detect pathogens in the aerosol samples collected by the air treatment devices 102 for generation of a pathogen status for each corresponding indoor environment. The pathogen status can be associated with the presence, absence, exposure risk, and/or other information relating to pathogens in the indoor environment. In some examples, part or all of the diagnostic system 104 can be remote from the air treatment devices 102 (e.g. at a remote site—for example, an external laboratory).

In some examples, part or all of the diagnostic system 104 can be integrated with the air treatment devices 102. For example, the diagnostic system 104 can include a plurality of diagnostic devices 150, with each air treatment device 102 including a corresponding diagnostic device 150 operable to detect pathogens in aerosol samples collected by corresponding aerosol collection units 132. In some embodiments, each diagnostic device 150 can include a pathogen detector adjacent a corresponding aerosol collection unit 132 for detecting likelihood of the presence of one or more pathogens in the aerosol sample of the aerosol collection unit 132, which can be used to determine a pathogen status associated with that air treatment device 102 and its corresponding indoor environment. The pathogen detector can be, for example, a particle detector and/or an antigen detector for detection of specific pathogens. In some embodiments, the testing for presence of pathogens by the diagnostic device 150 can be performed continuously and/or intermittently, and can be automated or performed manually by an operator at selected time intervals.

In some examples, diagnostic devices 150 integrated with corresponding air treatment devices 102 can offer simple testing for likely presence of pathogens, and further testing (e.g. more sophisticated analysis for pathogen variant type, pathogen concentration, etc.) may be desirable and conducted by external devices of the diagnostic system 104. The air treatment devices 102 (or integrated diagnostic devices 150 themselves) may be operable to automatically notify user(s)/operator(s) that corresponding aerosol collection units 132 should undergo further testing, such as for a specific targeted virus or virus variant. Such notification can be in response to specific environmental triggers (e.g., detection of an antigen by the diagnostic device, bad outbreak of a pathogen, etc.) or at predefined time periods (e.g., during and/or after an event taking place in the indoor environment, etc.). In some examples, when the diagnostic device 150 at an air treatment device 102 detects the likely presence of a pathogen (e.g. through the particle detector and/or antigen detector), that air treatment device 102 (or the diagnostic device 150 itself) can generate an alert to indicate that further analysis of the aerosol sample is required (e.g. at an external site with more sophisticated diagnostic equipment). In some circumstances, such as during a bad outbreak of a pathogen, all aerosol collection units 132 within proximity to an area associated with the outbreak may be flagged for further analysis at an external site. The air treatment devices 102 may also be operable to generate alerts to indicate that the filtration unit 126 (if any is included in the air treatment device 102) may need to be changed as well (e.g. in response to detection of pathogens).

In some examples, the process for external testing can include removing the flagged/used aerosol collection unit 132 from a corresponding air treatment device 102 (and optionally replacing it with a fresh aerosol collection unit), and storing the used aerosol collection unit 132 (or just the collection medium 134) in a transport medium (e.g., an envelope). The aerosol collection unit 132 can then be delivered to a diagnostic site, which can be located at a laboratory and/or other external site (e.g. near or remote from corresponding air treatment devices 102). At the diagnostic site, a pathogen status associated with the aerosol collection unit 132 (and the air treatment device 102 from which it was removed) can be determined based on, for example, nucleic acid sequences in the aerosol sample. For example, Deoxyribonucleic Acid (DNA) and/or Ribonucleic Acid (RNA) can be extracted from the aerosol collection unit 132 and amplified with various techniques such as, for example, loop-mediated isothermal amplification (LAMP), Polymerase Chain Reaction (PCR), and/or Reverse Transcriptase PCR (RT-PCR). Amplified DNA and/or RNA extracted from the aerosol collection unit 132 can then be analyzed for the presence of specific pathogens (e.g. viruses and/or specific viral variants). This can be performed with a variety of methods including, for example, the use of RNA and/or DNA probes, which in some embodiments, can be used in a multiplexed arrangement. It may be possible that more than one virus or infectious element can be identified from the same aerosol sample and more than one specific variant can be identified with these techniques. The further testing may also estimate relative quantity and/or concentration of pathogens, including viruses and/or microbes including bacteria.

In some examples, the diagnostic device 150 at the air treatment device 102 can be configured to perform more sophisticated automated testing based on nucleic acid sequences. For example, the diagnostic device 150 can include one or more automated testing cartridges configured to automatically extract and amplify DNA and/or RNA from the aerosol collection unit 132. This can include exposing the aerosol collection unit to lysis solution and/or other fluids (which can be stored in releasable fluid cartridges or capsules or in a reservoir in the air treatment device 102) for breaking down cell walls to release nucleic acids. The fluids can include markers or amplifier molecules that, when heated by a heater of the diagnostic device 150, fluoresce in the presence of select pathogens. A camera can be positioned to capture images of the markers. The captured images can be analyzed by a processor at the air treatment device 102, or transmitted through network 108 to the monitoring system 106 (or another system component) for further analysis, to determine a pathogen status associated with that air treatment device 102 and corresponding indoor environment. In some examples, such nucleic acid based testing can be triggered automatically at pre-defined intervals or in response to environmental triggers (e.g. detection of the likelihood of pathogen presence by an antigen detector of the diagnostic device).

The diagnostic system 104 can include at least one processor, memory, and a communication interface operable to connect the system 104 (and/or corresponding diagnostic devices 150) to the network 108 for communication with other system components (e.g. through a wired or wireless connection).

Referring to FIG. 1, the network 108 may be any network capable of carrying data, including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these, capable of interfacing with, and enabling communication between the components described herein.

The monitoring system 106 includes a processor and memory. The processor may be any suitable processors, controllers, or digital signal processors that can provide sufficient processing power depending on the configuration, purposes and requirements of the monitoring system 106. The memory can include RAM, ROM, one or more hard drives, one or more flash drives or some other suitable data storage elements such as disk drives, etc. The memory can include one or more databases (not shown) for storing information relating to the operation of the air treatment devices 102 and/or diagnostic system 104, such as data related to the aerosol samples, pathogen status, and/or user tracking data, and information relating to visual representations generated based on the aerosol sample data, pathogen status, and/or user tracking data. The monitoring system 106 can include one or more computer servers that may be distributed over a wide geographic area and connected via the network 108.

The monitoring system 106 can communicate with the air treatment devices 102 and/or diagnostic system 104 to generate a visual representation for visually illustrating the safety status of the indoor environments where the air treatment devices 102 are (or were) located during collection of aerosol samples. The monitoring system 106 can, in some embodiments, include in the visual representation other data collected by other devices related to the individuals being monitored by the air treatment devices 102 and/or environmental factors, such as but not limited to, weather information (e.g., humidity levels, pressure, temperature, etc.), any power outage instances (e.g., HVAC may not be clearing air properly), and public transport routes, etc. The additional data can augment the functionality of the visual representations as well as improve the accuracy of any risk and exposure predictions made by the monitoring system 106 with reference to the visual representations.

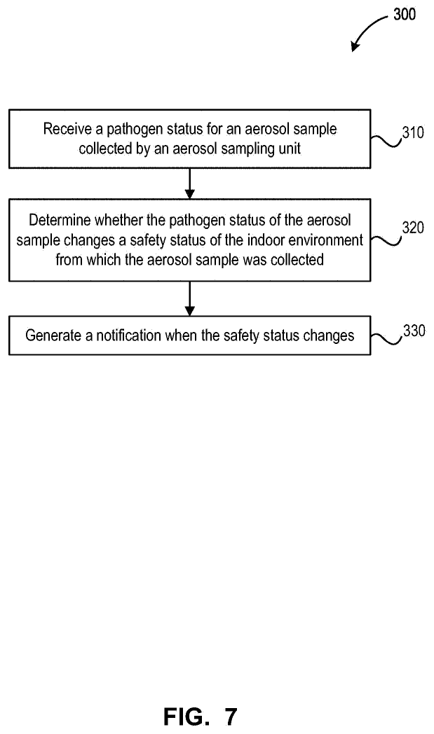

Referring now to FIG. 7, a flowchart 300 is shown illustrating steps for monitoring the indoor environments for aerosolized pathogens by the monitoring system 106. At 310, the monitoring system 106 receives a pathogen status for an aerosol sample collected by an aerosol collection unit 132 of a corresponding air treatment device 102. The monitoring system 106 can receive the pathogen status from the diagnostic system 104 via the network 108 (e.g. from the air treatment devices 102 via the network 108 if the testing occurs at the air treatment devices 102, from a remote computing device of the diagnostic system 104 via the network 108, and/or from entry of pathogen status data into the monitoring system 106).

At 320, the monitoring system 106 determines whether the pathogen status of the aerosol sample changes a safety status of the indoor environment from which the aerosol sample was collected. For example, when the pathogen status of the aerosol sample is determined as positive (indicating, for example, that one or more pathogens are likely present), the monitoring system 106 can determine that the safety status of the indoor environment has changed by comparing whether the existing safety status is positive or negative (indicating, for example, that the one or more pathogens are likely not present). In some embodiments, the monitoring system 106 can determine that the safety status needs to change only when the pathogen status indicates that the associated risk of exposure has exceeded, or fallen below, a risk level associated with the safety status currently assigned to the indoor environment. For example, the safety status can be organized in two or more levels, with each level being associated with pathogen detection, a range of pathogen quantity and/or concentration, and/or detection frequency.

In some examples, each air treatment device 102 can include a sensor system having one or more environmental sensors for sensing environmental conditions associated with pathogen exposure risk of the indoor environment. The environmental conditions can include, for example, temperature, humidity, and/or barometric pressure. The environmental sensor data relating to the environmental conditions can be transmitted to the monitoring system 106. The processor of the monitoring system 106 can be operable to determine whether to change or adjust the safety status for the indoor environment based at least in part on the environmental conditions. For example, the monitoring system 106 can determine whether to change or adjust the present (or a future) safety status based on a predictive algorithm associating the environmental conditions with pathogen exposure risk (e.g. associating a drop in temperature or humidity below corresponding thresholds as indicative of increased pathogen exposure risk [e.g. increased pathogen lifetime, increased contagiousness, and/or decreased aerosol droplet size], and/or pressure changes with a risk of an increase in the circulation of aerosolized pathogens).

At 330, in response to determining a change in the safety status, the monitoring system 106 generates a notification associated with the change in the safety status for the indoor environment. Generating the notification can include, for example, initiating an update of a visual representation of the indoor environment based on the safety status change. The visual representation can comprise, for example, a portion of a map corresponding to a location of the indoor environment. The map can include topographic features indicative of an elevation of the location, and can be, for example, a reference (e.g. geographical, topographical, and/or political) map, and can be a dynamic map. In examples with multiple air treatment devices at different locations, the visual representation can include portions of the map corresponding to each of the different locations, to produce a dynamic “bio-weather” map showing pathogen-related information for a plurality of locations. In some examples, the visual representation can comprise a three-dimensional representation of a location of the indoor environment (e.g. within three-dimensional space of a building). In some examples, the visual representation can comprise, for example, a floor plan view or dynamic three-dimensional models. In some examples, the visual representation can be presentable through an augmented reality system to inform users of any status changes within indoor environment as they walk within or near the environments.

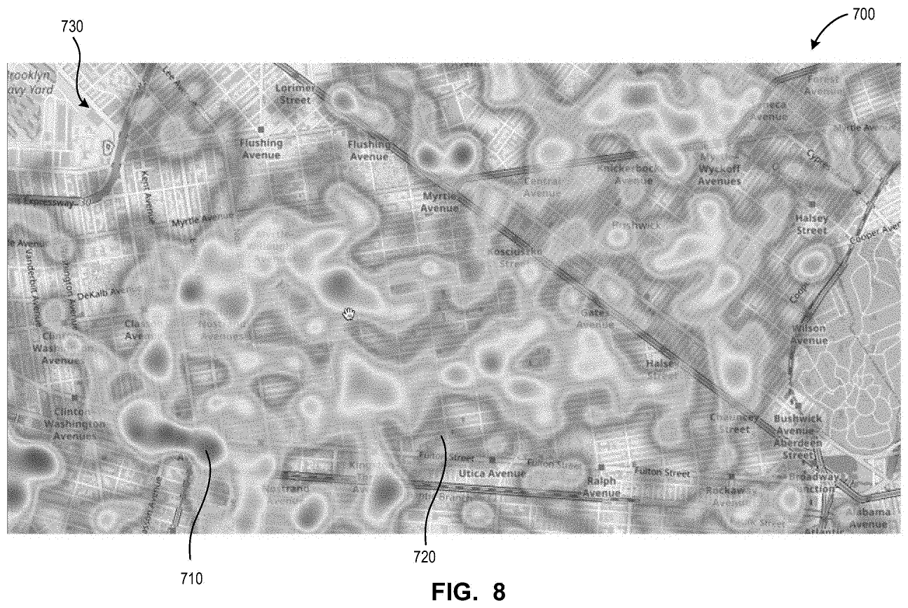

FIG. 8 is an example visual representation 700 generated by the monitoring system 106 for displaying pathogen status of various indoor environments. The visual representation 700 takes the form of a dynamic reference “bio-weather” map with a multi-colored overlay representing the safety status of various indoor environments. For example, area 710 is represented by a dark red color indicative of a relatively high risk area; area 720 is represented by a light blue color indicative of a relatively low risk area, and area 730 with no colored overlay is an unaffected or unmonitored area.

The visual representations can assist individuals to plan ahead when needing to visit areas with evidence of pathogens, as well as to aid in the study of the transmissibility of various pathogens as it may relate to various environmental factors.

The notification by the monitoring system 106 when the safety status changes can include, e.g., a listing of areas that should be approached with caution and/or avoided, and can also identify safety protocols for each monitored location (e.g., PPE requirements, such as mask, face shield, full biohazard suit requirements, etc.).

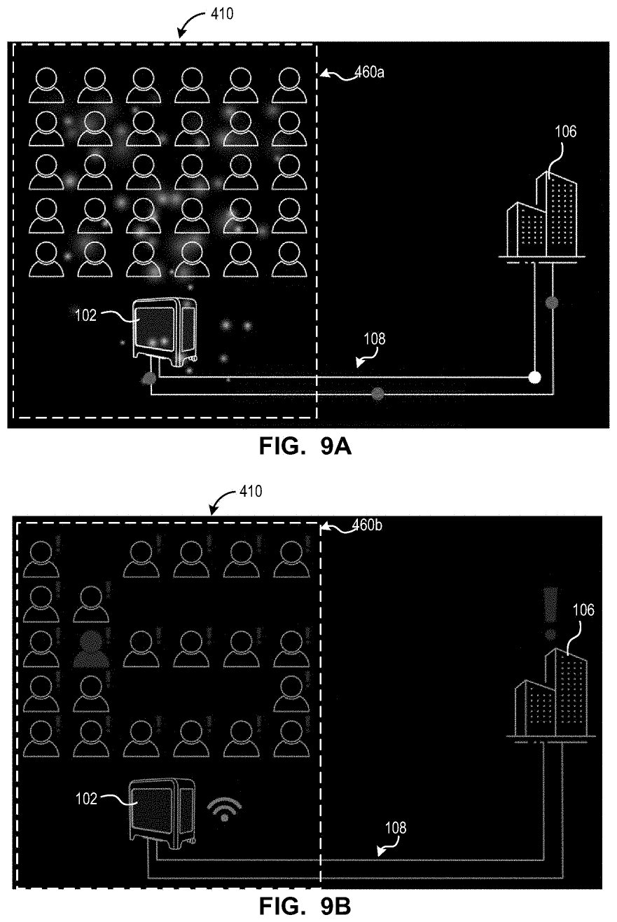

The monitoring system 106 can, in some embodiments, conduct electronic contact tracing to track individuals who may have been affected by a change in safety status (e.g. through pathogen exposure) at or near the monitored indoor environments. Reference will now be made to FIGS. 9A and 9B, which illustrates an example of the system 100 in use for contact tracing.

FIG. 9A shows an air treatment device 102 collecting aerosol samples from within the indoor environment 410, in which a group of individuals 460a are located or have visited previously. The group of individuals 460a are shown for purpose of illustration to be grouped together but it will be understood that the individuals within the group 460a could have been within the indoor environment and/or in proximity of the air treatment device 102 at different times. In the illustrated example, the air treatment device 102 would have collected one or more aerosol samples that provide information regarding pathogen exposure risk for individuals in the group 460a.

The air treatment device 102 is in communication with the monitoring system 106 via network 108. The monitoring system 106 can receive the pathogen status for the aerosol sample (e.g. from the air treatment device 102 or from the diagnostic system 104 via the network 108). When the monitoring system 106 determines that the pathogen status of the indoor environment 410 has changed to, for example, a positive pathogen status, the monitoring system 106 can identify individuals potentially affected by the positive pathogen status from the user tracking data collected by the air treatment device 102. As illustrated in FIG. 9B, the individuals affected by the positive pathogen status can be a subset 460b of the group 460a. The subset 460b may be those individuals who were in proximity to the air treatment device 102 for or within a predetermined time period of when the aerosol sample was collected by the aerosol collection unit 132. The monitoring system 106 can then generate a notification in the form of an alert (e.g. a risk of pathogen exposure alert) to the subset 460b of individuals (e.g. to their mobile phones) regarding the change in the safety status (e.g. advising of a risk of pathogen exposure).

In some embodiments, the monitoring system 106 can make predictions on future safety status of the various indoor environments based on the available historical data—such as the data available from the visual representations 700, user behavior data collected by the air treatment devices 102, and/or present or expected environment conditions of the indoor environments. For example, the monitoring system 106 can identify patterns by analyzing where individuals typically congregate and meet, and where sick individuals typically congregate and meet, and patterns associated with known high-risk groups (e.g., based on profession, socioeconomic background, etc.). The monitoring system 106 can provide recommendations to users and organizations using the air treatment devices 102.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

1. A pathogen surveillance system configured to monitor indoor environments for aerosolized pathogens, comprising:
a) one or more air treatment devices for treating air in corresponding indoor environments, each air treatment device including:
i) at least one air flow passage extending through the device;
ii) an air circulation unit operable to urge flow of air through the air flow passage;
iii) an air filtration unit in the air flow passage for filtering the air flowing through the air flow passage;
iv) a sterilization unit operable to sterilize the air flowing through the air flow passage; and
v) an aerosol collection unit configured to collect an aerosol sample from the air flowing into the air flow passage; and
b) a monitoring system comprising a processor operable to:
receive a pathogen status for the aerosol sample collected by the aerosol collection unit;
determine whether the pathogen status of the aerosol sample changes a safety status of a corresponding indoor environment from which the aerosol sample was collected; and
in response to determining a change in the safety status, generate a notification associated with the change in the safety status for the indoor environment.

2. The system of claim 1, wherein generating the notification comprises updating a visual representation of the indoor environment based on the safety status change.

3. The system of claim 2, wherein the visual representation comprises a portion of a map corresponding to a location of the indoor environment.

4. The system of claim 3, wherein the map comprises topographic features indicative of an elevation of the location.

5. The system of claim 2, wherein the one or more air treatment devices include a plurality of the air treatment devices positionable in different locations, and wherein the visual representation comprises portions of a map corresponding to the different locations.

6. The system of claim 2, wherein the visual representation comprises a 3-dimensional representation of a location of the indoor environment within a building.

7. The system of claim 1, wherein each air treatment device comprises a user tracking unit for collecting user tracking data associated with individuals within a detection range of the air treatment device.

8. The system of claim 7, wherein generating the notification comprises:
identifying one or more individuals affected by the change in the safety status for the indoor environment based on the user tracking data collected by the user tracking unit; and
generating an alert for transmission to the one or more individuals regarding the change in the safety status.

9. The system of claim 1, further comprising a diagnostic system operable to detect pathogens in the aerosol sample for generation of the pathogen status.

10. The system of claim 9, wherein each air treatment device includes a corresponding diagnostic device of the diagnostic system, the diagnostic device operable to detect pathogens in the aerosol sample collected by the air treatment device.

11. The system of claim 10, wherein the diagnostic device comprises at least one of a particle detector and an antigen detector.

12. The system of claim 9, wherein the diagnostic system is operable to detect pathogens based on nucleic acid sequences.

13. The system of claim 12, wherein the filtration unit comprises one or more air filters extending across the air flow passage.

14. The system of claim 1, wherein the sterilization unit is operable to sterilize the filtration unit.

15. The system of claim 1, wherein the aerosol collection unit is upstream of the filtration unit.

16. The system of claim 1, wherein the aerosol collection unit comprises an aerosol capture medium extending only partially across the air flow passage to permit air flow around the aerosol collection unit.

17. The system of claim 1, wherein the aerosol collection unit comprises a plurality of aerosol sampling elements configured to collect corresponding aerosol samples at different times.

18. The system of claim 1, wherein the monitoring system receives the pathogen status for the aerosol sample from the one or more air treatment devices via a network.

19. The system of claim 1, wherein the monitoring system receives the pathogen status for the aerosol sample from a remote computing device via a network.

20. The system of claim 1, wherein the air circulation unit comprises one or more fans.

21. The system of claim 1, wherein the sterilization unit comprises an ultraviolet C (UVC) source for UVC irradiation.

22. The system of claim 1, wherein each air treatment device is mobile to facilitate movement to different locations.

23. The system of claim 1, wherein each air treatment device comprises a processor and a drive system operable by the processor to enable autonomous movement of the air treatment device.

24. The system of claim 1, further comprising a mobile duct system including one or more ducts connectable between an outlet of the air flow passage and an external environment outside the indoor environment for negatively pressurizing the indoor environment.

25. The system of claim 1, wherein the one or more air treatment devices comprise a plurality of air treatment devices, and each air treatment device comprises a wireless communication unit to enable wireless data transfer and automated coordination of the plurality of air treatment devices.

26. The system of claim 1, wherein each air treatment device includes a sensor system having one or more environmental sensors for sensing environmental conditions associated with pathogen exposure risk of the indoor environment, and wherein the processor of the monitoring system is operable to determine whether to change the safety status for the indoor environment based at least in part on the environmental conditions.

27. The system of claim 26, wherein the environmental conditions include at least one of temperature, humidity, and barometric pressure.

28. A method of monitoring indoor environments for aerosolized pathogens, comprising:
a) receiving a pathogen status for each of a plurality of aerosol samples collected by a plurality of corresponding air treatment devices;
b) determining whether the pathogen status for each aerosol sample changes a safety status of a corresponding indoor environment from which the aerosol sample was collected; and
c) in response to determining a change in the safety status, generating a notification associated with the change in the safety status for the corresponding indoor environment.

29. The method of claim 28, wherein generating the notification comprises updating a visual representation of the corresponding indoor environment based on the safety status change.

30. The method of claim 29, wherein the visual representation comprises a portion of a map corresponding to a location of the corresponding indoor environment.

31. The method of claim 28, wherein each air treatment device comprises a user tracking unit for collecting user tracking data associated with individuals within a detection range of the air treatment device, and wherein generating the notification comprises:
identifying one or more individuals affected by the change in safety status based on user tracking data collected by a user tracking unit of the air treatment device; and
generating an alert for transmission to the one or more individuals, the alert advising the one or more individuals of the change in the safety status.

32. The method of claim 28, further comprising determining the pathogen status through testing of the aerosol sample based on nucleic acid sequences.

33. The method of claim 28, further comprising, for each air treatment device, urging flow of air from the corresponding indoor environment through an aerosol collection unit positioned in an air flow passage of the air treatment device to collect the aerosol sample, and sterilizing the air flowing through the air flow passage downstream of the aerosol collection unit.

34. A monitoring system for aerosolized pathogens comprising a processor operable to:
a) receive a pathogen status for each of a plurality of aerosol samples collected by a plurality of corresponding air treatment devices;
b) determine whether the pathogen status for each aerosol sample changes a safety status of a corresponding indoor environment from which the aerosol sample was collected; and
c) in response to determining a change in the safety status, generate a notification associated with the change in the safety status for the corresponding indoor environment.

35. The method of claim 34, wherein generating the notification comprises updating a visual representation of the corresponding indoor environment based on the safety status change.

36. The method of claim 35, wherein the visual representation comprises a portion of a map corresponding to a location of the corresponding indoor environment.
